(19) United States
(12) Patent Application Publication
(10) Pub. No.: US 2025/0135237 A1
(43) Pub. Date: May 1, 2025

(54) ULTRASOUND BEAN SHAPING

(71) Applicant: Oxford University Innovation Limited, Oxford (GB)

(72) Inventors: Luke RICHARDS, Oxford (Oxfordshire) (GB); Robin CLEVELAND, Oxford (Oxfordshire) (GB); Eleanor STRIDE, Oxford (Oxfordshire) (GB)

(21) Appl. No.: 18/681,881

(22) PCT Filed: Jul. 25, 2022
(86) PCT No.: PCT/GB2022/051942

§ 371 Date: Feb. 7, 2024

(30) Foreign Application Priority Data
Aug. 9, 2021 (GB) 2111427.7

(51) Int. Cl. A61N 7/00; B06B 1/04

(52) U.S. Cl. CPC A61N 7/00; B06B 1/04; A61N 2007/0056

(57) ABSTRACT

An ultrasound beam shaping device, and method of designing an ultrasound beam shaping device are disclosed. The beam shaping device comprises an array of waveguides formed in a structure that is capable of reflecting ultrasound. The lengths and cross-sections of the waveguides of the array are selected based on a desired shape of the ultrasound beam at a target, such that the amplitude and phase of the ultrasound emitted from the waveguides of the array provides the desired shape of the ultrasound beam at the target.

The present invention relates to beam shaping devices for shaping ultrasound beams, to methods of designing such beam shaping devices, and to methods of using such beam shaping devices.

In some applications it is desirable to shape an ultrasound beam to provide a desired sound/pressure field at a target. Conventionally this may be achieved with an array of ultrasound transducers, with the phase and/or amplitude of each source controlled to generate the desired beam shape. However, this requires complicated control circuitry. As a result, such ultrasound transducers arrays are expensive.

Ultrasound can be used therapeutically, for example in the treatment of cancer. Tissue inhomogeneity can affect the accuracy with which ultrasound can be focused in the body. This is problematic for example in applications in which high intensity ultrasound is used, such as therapeutic ablation. Bone causes the greatest phase aberration because of the large difference between its sound speed and those of water or soft tissue, but sound speed differences between soft tissues can also reduce the effectiveness of treatments.

In existing clinical systems, any phase corrections are typically made using ultrasound arrays, but there has been recent interest in using acoustic lenses to make phase corrections. Acoustic lenses could offer a more compact and affordable system than an equivalent array, particularly when phase corrections require a high spatial resolution and therefore a large number of elements, and associated driving electronics.

An acoustic lens consists of a material with a different sound speed from the propagating medium, enabling a change in the phase of the transmitted wave. An ideal material for an acoustic lens would be easy to form into complex shapes, have low acoustic attenuation, a sound speed significantly different from that of water, but a similar characteristic acoustic impedance to that of water to minimize reflection at the lens interface.

Recent studies investigating customized acoustic lenses using 3D printing technology have used silicone, PLA or proprietary polyjet resins. Silicone has a sound speed of 1030 m/s, and an impedance of 1.06 MRayl. The transmission of a silicone lens is excellent due to the similar impedance to water, and attenuation of just 0.7 dB/cm at 500 kHz. Construction requires casting, which slows production and may be less repeatable unless mixing and degassing are tightly controlled. PLA has a sound speed of approximately 1818 kg/m3 and acoustic impedance of approximately 2.05 MRayl. PLA is normally printed using Fused Deposition Modeling (FDM) that will likely leave voids, which could lead to poor acoustic properties. Polyjet printers can print with a large number of polymers with high accuracy. Polyjet printers are relatively fast compared to other 3D printing technologies, but more expensive, and time is also required to finish lenses e.g. to remove support structures.

According to a first aspect of the invention there is provided a method of designing a beam shaping device for shaping an ultrasound beam from an ultrasound source, the beam shaping device comprising an array of waveguides formed in a structure that is capable of reflecting ultrasound, the method comprising: determining a desired shape of the ultrasound beam at a target; selecting lengths and cross-sections of the waveguides of the array based on the desired shape of the ultrasound beam and on an ultrasound field produced by an ultrasound source such that the amplitude and phase of the ultrasound emitted from the waveguides of the array provides the desired shape of the ultrasound beam at the target.

This method allows beam shaping devices to be designed to provide a desired field at a target, without using an array of ultrasound sources. The waveguides of the beam shaping device provide a plurality of ultrasound paths, each with a controlled phase and amplitude adjustment, so that the outputs of the waveguides combine to form an ultrasound field with the desired shape at the target. The beam shaping device can be tailored to each individual target, and can be made of cheap, easily workable materials to provide for fast and easy manufacture. The beam shaping device can control both phase and amplitude of the ultrasound field, unlike acoustic lenses, providing greater control of the ultrasound field at the target.

The lengths and cross-sections of each waveguide are selected based on the amplitude and/or phase of ultrasound exiting that waveguide required to form the desired field at the target. As used herein, cross-section includes both cross-sectional shape and cross-sectional size. The cross-section may be the cross-section of a waveguide at the entrance to the waveguide, in a plane substantially orthogonal to a direction of propagation of ultrasounds from the ultrasound source. In some embodiments, one of the cross-section and length is fixed for all waveguides. In other embodiments, both cross-section and lengths vary across the array. In some embodiments the lengths of the waveguides may be primarily selected based on a desired phase of ultrasound exiting the waveguides. In some embodiments, the cross-sections are be selected primarily based on a desired amplitude of ultrasound exiting the waveguides.

The waveguides may in particular be intended to be aligned or substantially aligned with a direction of propagation of the ultrasound beam from the ultrasound source.

In some embodiments, the waveguides are holes formed through the structure. Holes can be manufactured quickly and reliably, facilitating production of the beam shaping device.

In some embodiments, the waveguides are pillars of material capable of transmitting ultrasound. The material capable of transmitting ultrasound may be a plastic. Pillars may provide more accurate phase and/or amplitude control at higher beam intensities, and may offer more control over geometry of the waveguides than using holes. The pillars may be formed using 3D printing.

In some embodiments, the structure is a substrate. In particular, the substrate may be formed of foam, or other material comprising entrapped gas pockets. The gas pockets reflect ultrasound waves, and so the overall substrate is capable of reflecting ultrasound. Such embodiments may be particularly advantageously combined with holes acting as the waveguides, to provide a cheap and easy to manufacture device. Such a waveguide may be useful for tailor-made, disposable beam shaping devices. That is, the cost and ease of manufacture of such a beam shaping device may make it desirable to design and build a waveguide array for a specific, one-time use. For example, such a beam shaping device may be designed to target an area (e.g. tumour area) of a specific patient, and may be constructed in a hospital.

In some embodiments, the structure comprises gas held in a container. In some such embodiments the container comprises a membrane or thin sheet of gas-impermeable material holding the gas in place. The gas may be one gas or a mixture of gases. The gas may be air.

In some embodiments the waveguides have the same cross-sectional shape and have varying cross-sectional sizes across the array to provide varying cross-sections. Selecting the cross-section may comprise selecting a characteristic cross-sectional size of the waveguides. The characteristic cross-sectional size may be one or more of: a smallest cross-sectional size, a largest cross-sectional size, a diameter (for substantially circular cross-section waveguides) or a circumdiameter, i.e. diameter of a circle inscribed/circumscribed by the cross-section of the waveguide (for non-circular cross-section waveguides).

In some embodiments, a smallest cross-sectional dimension (e.g. diameter of a circular cross-section or smallest side of a square or rectangular cross-section), or a circumdiameter of each waveguide is in a range with a maximum value of λ, where λ is the wavelength of the ultrasound from the ultrasound source in the the waveguide. In some embodiments, the smallest cross-sectional dimension or circumdiameter of each waveguide is in a range with a minimum value of 0.6λ, where λ is the wavelength of the ultrasound from the ultrasound source in the waveguide. . . . It has been found that such ranges allow the ultrasound transmission through the waveguide to be varied between no transmission and maximum transmission.

In some embodiments the pitch or average pitch of the waveguides selected from a range with a maximum value of 2λ, or preferably 1.5λ, or more preferably 1.2λ, where Δ is the wavelength of the ultrasound from the ultrasound source in the waveguide. In some embodiments the pitch or average pitch is selected from a range with a minimum value of 0.7λ, or preferably λ, or more preferably 1.1λ, where λ is the wavelength of the ultrasound from the ultrasound source in the waveguide. As used herein pitch is the distance between the centre of one waveguide to the centre of its nearest neighbour (e.g. measured in a plane substantially orthogonal to the direction of propagation of incident ultrasound). Pitch may be constant for all waveguides, or may vary across the array. It has been found that such pitches between waveguides are optimal to provide maximum transmission through the beam shaping device.

In some embodiments the structure comprises a first layer and a second layer, wherein the waveguides are formed through each of the first layer and the second layer. In some such embodiments, the cross-sections of each waveguide may be constant through the two layers. This may be used to extend the effective lengths of the waveguides. This may be useful for example where it is easier to construct shorter waveguides in each array, either due to the nature of constructing the waveguides, or the available structure material.

In some embodiments, one or more waveguides have cross-sections varying along their length. In such embodiments, the structure may comprise a first layer and second layer, with waveguides formed through each layer, wherein the cross-section of at least one of the waveguides varies between the first layer and the second layer. Each waveguide may have a first length in the first layer and a second length in the second layer, and wherein for at least one waveguide the first length is different to the second length. The lengths and cross-sections of the waveguides in the first layer are selected based on a desired one of amplitude or phase of ultrasound at the target; and the lengths and cross-sections of the waveguides in the second layer are selected based on the other of amplitude or phase of ultrasound at the target. In particular, the lengths of waveguides one layer may be selected to yield a desired phase, and the cross-sections (in particular cross-sectional size) of waveguides the other layer may be selected to yield the desired amplitude. Using multiple layers in this way may facilitate manufacturing of the device.

In some embodiments the ultrasound source emits ultrasound with a frequency of 3 MHz or less. In some embodiments the ultrasound source emits ultrasound with a frequency of 0.3 MHz or more. The ultrasound source may comprise one or more ultrasound transducers.

The beam shaping device may be particularly advantageously used in medical treatments. In particular, in some embodiments the target is a human body or is within a human body. For example, the target may be a tumour, or the target may be a region of the body comprising one or more tumours. The easily manufactured beam shaping devices of the present invention allows an ultrasound wave to be shaped to target the specific patient, taking account of phase changes and/or amplitude changes caused by transmission of waves through the body of that patient. Tailoring the ultrasound wave to the individual patient may improve the effectiveness of the intended treatment for which the ultrasound is being used.

In accordance with a second aspect of the invention there is provided a computer program storing instructions which, when executed by a computer, cause the computer to perform the method of any embodiment of the first aspect.

In accordance with a third aspect of the invention there is provided a computer readable medium storing the computer program of the second aspect.

In accordance with a fourth aspect of the invention there is provided a method of forming a beam shaping device for shaping an ultrasound beam from an ultrasound source, the method comprising: providing a structure capable of reflecting ultrasound; performing the method of any embodiment of the first aspect to determine parameters of waveguides of a waveguide array; and forming the waveguide array in the structure based on the determined parameters.

In accordance with a fifth aspect of the invention there is provided beam shaping device for shaping an ultrasound beam from an ultrasound source, the beam shaping device comprising an array of waveguides formed in a structure that is capable of reflecting ultrasound, wherein the waveguides have lengths and cross-sections selected such that the amplitude and phase of the ultrasound emitted from the waveguides of the array provides a desired shape of an ultrasound beam at a target.

Details of the parameters, target, and ultrasound source discussed in relation the first aspect may also apply to the fifth aspect.

In accordance with a sixth aspect there is provided a method of shaping an ultrasound beam from an ultrasound source, the method comprising: positioning a beam shaping device according to any embodiment of the fifth aspect between the ultrasound source and a target such ultrasound from the ultrasound source is incident upon the waveguide array of the beam shaping device, and such that ultrasound exiting the waveguide array forms a desired ultrasound beam at the target. The target may particularly be the target for which the beam shaping device was designed.

Embodiments of the invention will now be described, by way of example only, with reference to the accompanying drawings in which corresponding reference symbols indicate corresponding parts, and in which:

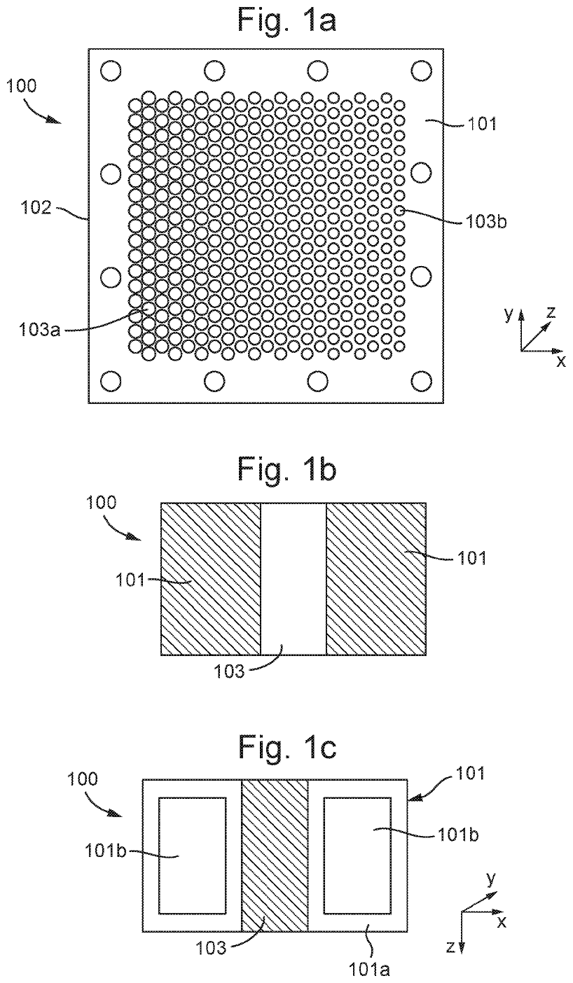

FIG. 1a is a schematic representation of a beam shaping device;

FIG. 1b illustrates a waveguide formed of a hole through a substrate;

FIG. 1c illustrates a waveguide formed of a pillar;

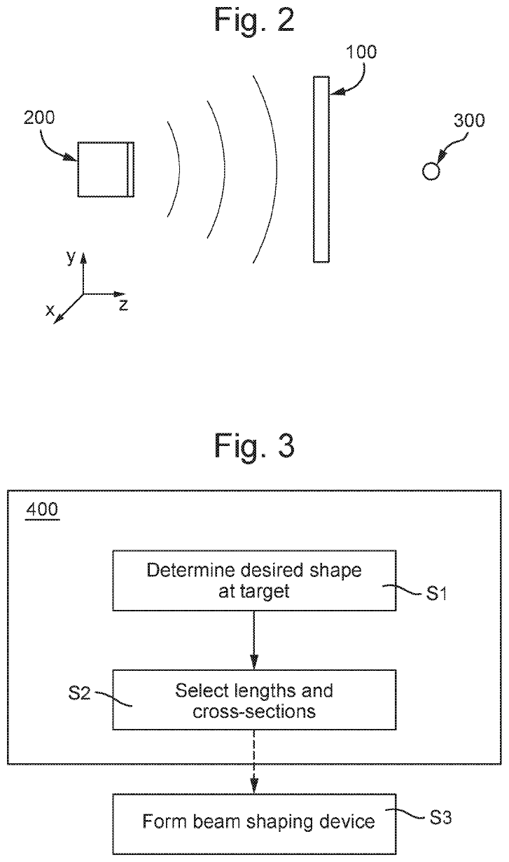

FIG. 2 illustrates use of a beam shaping device with a ultrasound source;

FIG. 3 illustrates a method of designing a beam shaping device;

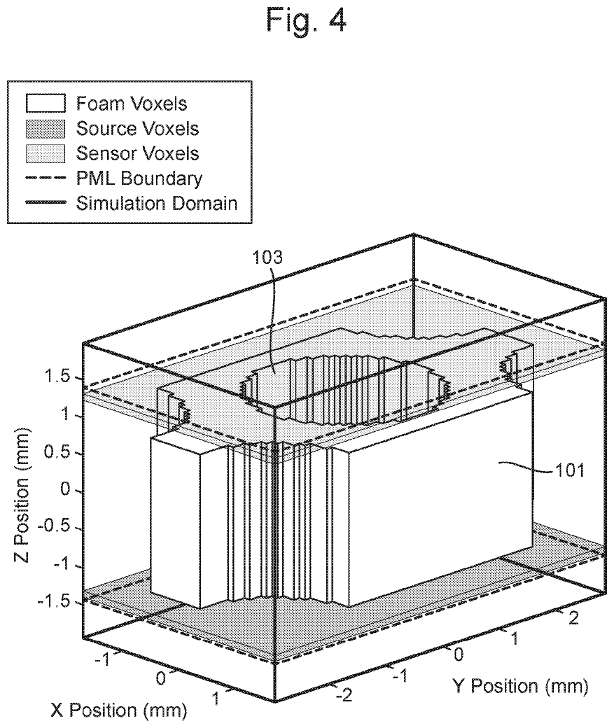

FIG. 4 shows a cell uses for numerical modelling of a beam shaping device;

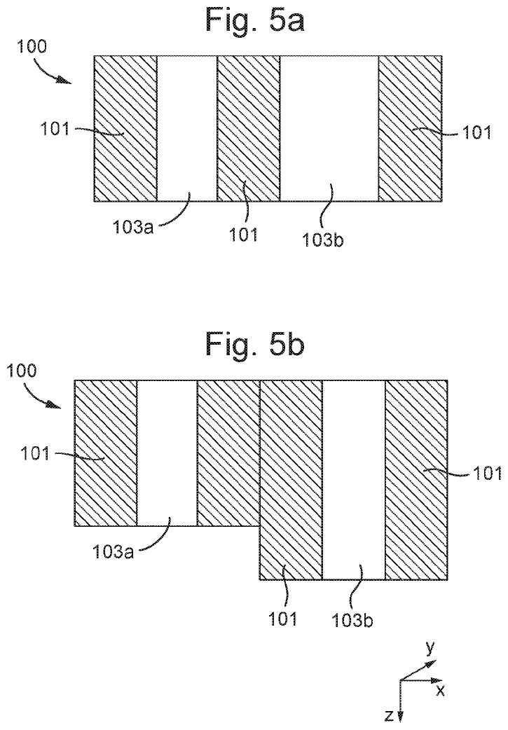

FIG. 5a illustrates waveguides of different cross-sections;

FIG. 5b illustrates waveguides of different lengths

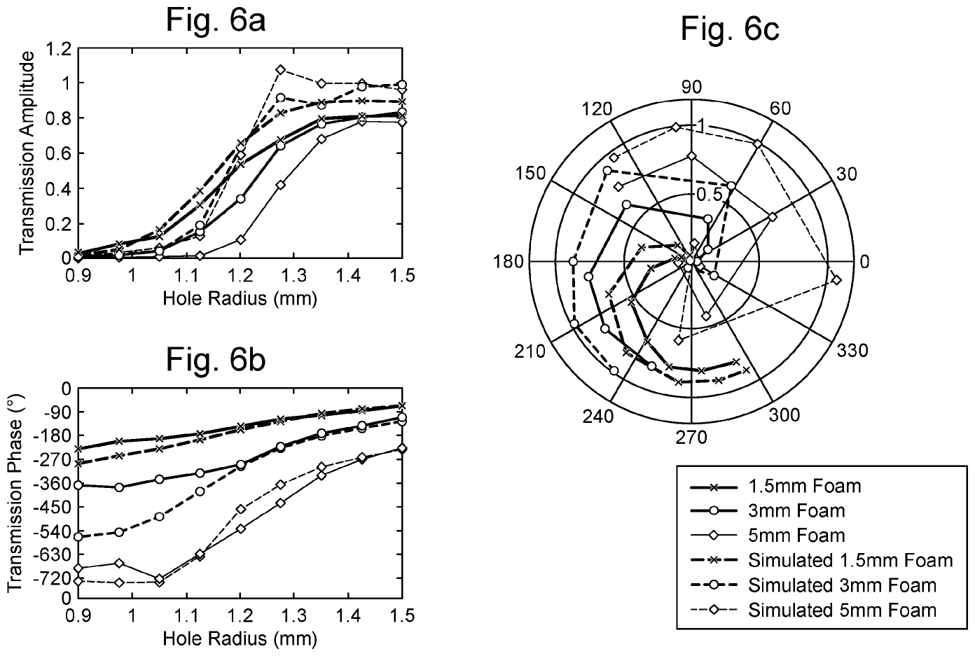

FIG. 6 illustrates modelled and experimental results for various beam shaping devices;

FIGS. 6a and 6b compare ultrasound amplitude with and without a beam shaping device;

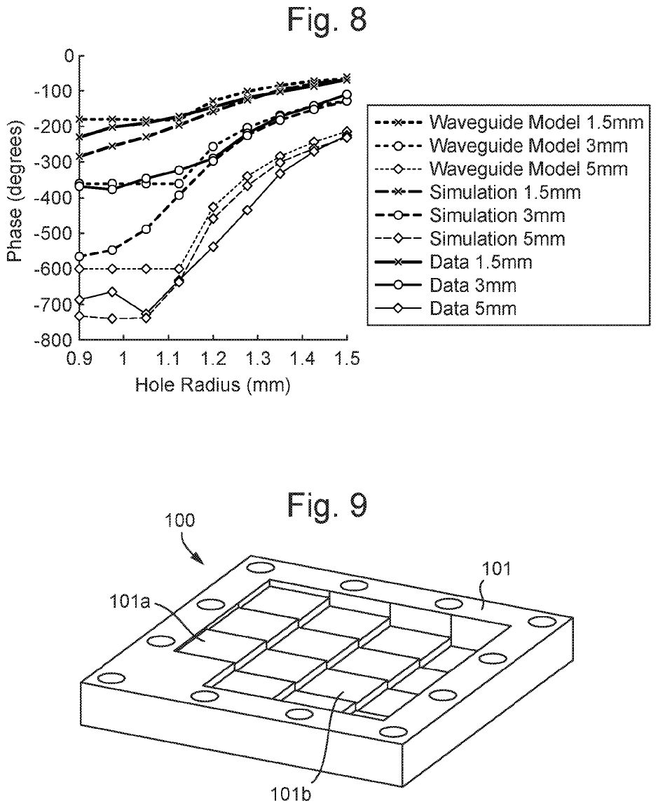

FIG. 8 illustrates further results of measurements on beam shaping devices;

FIG. 9 is a schematic representation of an acoustic lens constructed as a comparison to the beam shaping device;

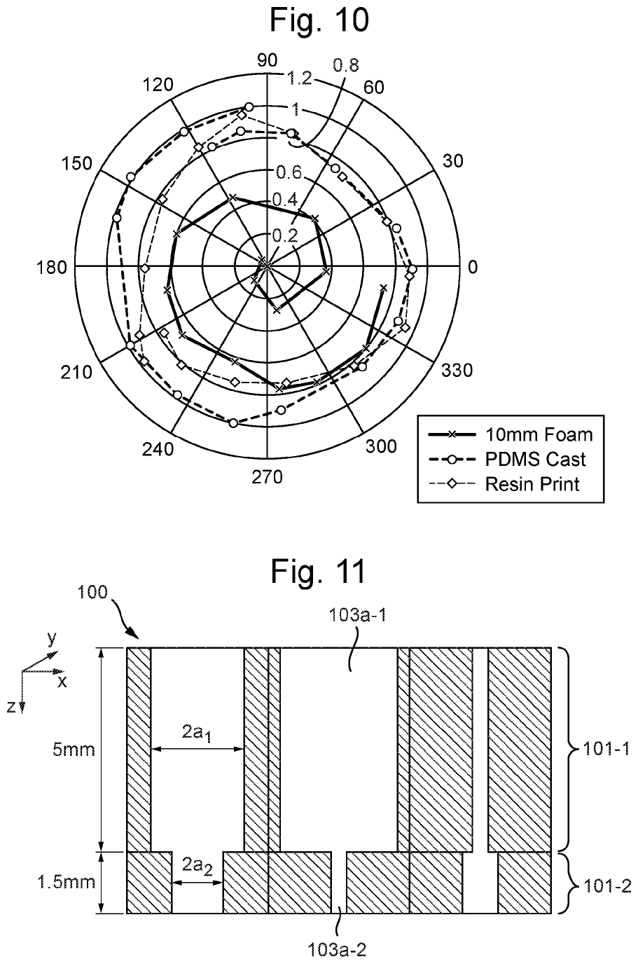

FIG. 10 shows results of a comparison between the beam shaping device and the acoustic lenses;

FIG. 11. is a schematic representation of a beam shaping device comprising multiple layers;

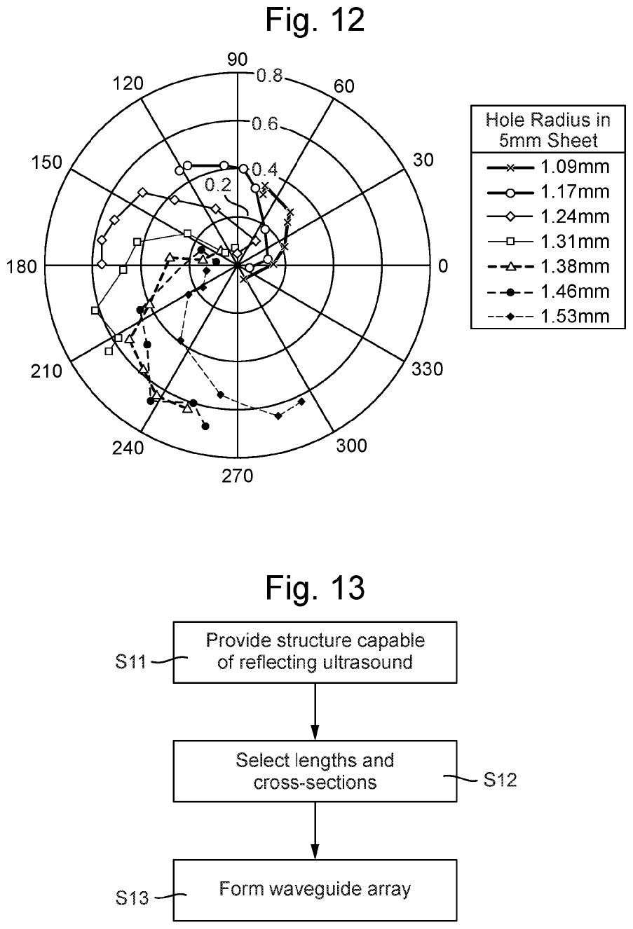

FIG. 12 illustrates results of measurements on the beam shaping device of FIG. 11; and

FIG. 13 illustrates a method of forming a beam shaping device.

FIG. 1a illustrates an example beam shaping device 100. Beam splitting device 100 is for shaping an ultrasound beam from an ultrasound source 200, an example of which is illustrated in FIG. 2. The ultrasound source 200 may take any form, and may emit ultrasound with a frequency of 0.3 MHz or more, and/or 3 MHz or less. More generally, the ultrasound used in accordance with this disclosure be in a range from 20 kHz to 10 GHz. The ultrasound source 200 may comprise one or more than one ultrasound transducers.

Beam shaping device 100 comprises a structure 101 that is capable of reflecting ultrasound. An array 102 of waveguides 103 is formed through the structure 101. For clarity, only two individual waveguides 103a, 103b are labelled in FIG. 1a. The larger holes around the perimeter of the structure 101 are optional mounting holes, which may be used to secure the device 100 in place when in use.

FIG. 2 illustrates a method of shaping an ultrasound beam from an ultrasound source, using a beam shaping device 100. As shown in FIG. 2, in use the beam shaping device is positioned between an ultrasound source 200 and a target 300. Ultrasound from the ultrasound source 200 propagates substantially along the z axis, and is incident upon first side of the device 100. The waveguides 103 of array 102 are arranged across a plane substantially orthogonal to the direction of propagation of the ultrasound (i.e. arranged in the x-y plane in FIG. 1a). The waveguides 103 provide paths for ultrasound to pass through the device 100, and thence onto the source 300. Each waveguide 103 is designed so that ultrasound exiting the waveguide has a particular phase and/or amplitude. Ultrasound exiting the waveguides 103 combines to form a desired ultrasound beam at the source 300.

The structure 101 of device 100 is capable of at least partially reflecting ultrasound incident upon it. This provides a block to incident ultrasound that does not enter a waveguide 103, allowing the ultrasound field after the device 100 to be controlled by appropriate selection of waveguide parameters. It also provides a reflective barrier at the waveguide/structure interface to maintain ultrasound within the waveguides 103. For example, the structure 101 may reflect 80% or more, or 90% or more, or 95% or more or preferably 99% or more of ultrasound (e.g. from the ultrasound source 200) incident upon it. The structure 101 may reflect ultrasound incident substantially orthogonally to the surface of the structure 101, and/or ultrasound incident at an acute angle to the surface of the structure 101. In particular, the structure 101 is such that ultrasound passing through a waveguide 103 and incident at a boundary of that waveguide 103 and the structure 101 is partially or totally reflected.

The beam shaping device 100 may be designed for use in an ultrasound transmission medium, such as water. The structure 101 may be capable of reflecting ultrasound travelling within that transmission medium. In particular, the ratio of acoustic impedance of the structure 101 to the acoustic impedance of the transmission medium is 1/100 or less, or 1/500 or less. In general, the structure 101 may have an acoustic impedance of 20000 Rayl or less, or 15000 Rayl or less (where 1 Rayl is 1 kg/m²/s). The structure 101 may have an acoustic impedance of 400 Rayl or more, or 1000 Rayl or 1800 Rayl or more. For example the acoustic impedance of a structure 101 formed of an air-filled container, such as that in FIG. 1b, may have an acoustic impedance of approximately 420 Rayl, matching the acoustic impedance of air. As used herein, acoustic impedance may refer to specific acoustic impedance. Acoustic impedance may be measured in any known way, such as the single microphone method in an impedance tube. Measurements of acoustic impedance may be performed at standard temperature and pressure.

The structure 101 may be a substrate, i.e. formed of a substantially rigid material, and providing a first surface and second surface (surfaces in the x-y plane in FIG. 1a). The waveguide array 102 is formed through the substrate, to provide paths for ultrasound to pass from the first surface to the second surface. FIG. 1b illustrates a cross-section through part of a device 100, showing one of the plurality of waveguides 103. In this case the waveguide 103 is a hole, formed through the substrate of structure 101. In use, a transmission medium in which the ultrasound propagates (e.g. water) fills the hole, providing a path for the ultrasound. Holes are very easy to form, so this provides a fast and cheap way of forming the waveguide array.

Advantageously, the substrate forming structure 101 may be a foam. A foam comprises pockets of air. Ultrasound waves can reflect from the pockets of air, making the foam structure 101 overall capable of reflecting ultrasound. Foam is a particularly cheap, readily available, and easily tooled material. Holes as waveguides 103 in a foam structure 101 may provide a particularly cheap and easily made device, with the benefits of tailoring the beam shape to that desired at the target 300. The theoretical and experimental results presented below demonstrate the feasibility of manipulating ultrasound waves using an array 102 of holes cut a foam sheet and suspended in water as a transmission medium. The water fills the holes, allowing ultrasound propagate through the holes. Any transmission through the foam is negligible due to the large impedance difference between the foam structure 101 and the water.

An alternative structure 101 is illustrated in FIG. 1c. FIG. 1c illustrates a cross-section through part of a device 100, showing one of the plurality of waveguides 103. In this case, the structure 101 comprises a container 101a holding a gas 101b. The container may be a membrane, substantially impermeable to the gas, which holds the gas in place around the waveguides 103 of the waveguide array 102. The gas may be air. The waveguides 103 may be holes providing passage through the structure 101. In the example of FIG. 1c, however, the waveguides 103 (or a subset thereof) are formed of pillars of material that is capable of transmitting ultrasound, such as a plastic. Using such pillars as waveguides 103 may provide more accurate control of the amplitude and/or phase of ultrasound exiting the waveguide 103 than holes alone.

In general, any suitable combination of structure 101 and waveguides 103 may be used. For example, pillars capable of transmitting ultrasound may be inserted into holes formed through a substrate to provide the waveguides 103.

The waveguide array 102 may comprise any number of waveguides, for example one or more, two or more, 5 or more, 10 or more, 20 or more, 50 or more. The number of waveguides, spacing between waveguides (referred to as pitch herein), and cross-section (in the x-y plane) of the waveguides will determine the overall proportion of incident ultrasound that travels through the beam shaping device 100. The properties of each individual waveguide 103 will determine the phase and amplitude of ultrasound exiting that waveguide 103. This can be used to design a beam shaping device 100 to provide a desired ultrasound field at a target 300.

In the example shown in FIG. 1a, the waveguides 103 have a cross-sectional shape that is circular, but the waveguides of the present invention may in general have any shape, including also square or rectangular.

FIG. 3 illustrates a method of designing a beam shaping device 100 for shaping an ultrasound beam from an ultrasound source 200. The method comprises designing steps S1 and S2, and optional forming step S3. The method of design represented by steps S1 and S2 may be implemented as a computer program storing instructions which, when executed by a computer 400, cause the computer 400 to perform the method. The computer program may be stored on a computer readable medium (e.g. a non-transient computer readable medium). The computer readable medium may be part of computer 400, or external to computer 400.

The method of FIG. 3 starts at step S1, at which a desired shape of the ultrasound beam at a target 300 is determined. The desired shape may be an average phase and/or amplitude of the ultrasound wave at the target 300. The desired shape may include a beam width at the target 300. The desired shape may include a desired phase and/or amplitude at one or more spatial positions at the target 300. The spatial positions may be positions along the direction of propagation of the ultrasound wave (i.e. along z in FIG. 2). The spatial positions may be positions in a plane substantially orthogonal to the direction of propagation of the wave (i.e. the x-y plane in FIG. 2). The spatial positions may be positions in 3-dimensional space. In other words, the desired field at a point, or in 1, 2, or 3 dimensions may be determined. The desired shape of the beam at the target 300 may be determined by any appropriate method, as would be appreciated by the skilled person. For example, the desired shape may be determined based on measurements of the target 300, or a model of the target 300. The measurements/model may identify phase and/or amplitude changes of ultrasound incident on the target 300, caused either by the target 300 itself or by material in the beam path before the target 300.

The beam shaping device 100 may be particularly useful in medical applications. In particular, the target 300 may be a human body, or may be within a human body. Ultrasound therapy is used in the treatment of cancer. In some cases, a tightly focused beam is used to target a single tumour at a known location. In other cases, small tumours of unknown location are targeted with an ultrasound-activated agent. In these cases, a wide ultrasound field is desired to activate the agents. However, due to the phase changes and amplitude losses of ultrasound passing through the body, this conventionally requires large and expensive ultrasound transducers. Alternatively a multiplexed array of transducers can be used to create a field shape to correct for amplitude/phase changes, but again this is an expensive solution. The beam shaping device 100 of the present disclosure, especially when constructed of a low cost material such as foam, can provide the same ultrasound field shaping, without the cost. The beam shaping device 100 can be designed for an individual patient. Thus in some examples the method of FIG. 3 comprises taking and/or receiving measurements of or around the target. For example, where the target is in a patient, measurements may be received of the ultrasound adjusting properties of the tissue of the patient through which ultrasound will have to travel.

Other potential uses of the devices 100 disclosed herein include as a disposable grating placed on a large High Intensity Focused Ultrasound (HIFU) transducer for patient specific corrections for HIFU treatments in the abdomen or the skull. Applications such as hyperthermia driven drug delivery may also be possible. Non-medical uses may include for customized devices 100 for fields such as underwater imaging or non-destructive testing.

Having determined the desired beam shape at the target 300, the method of FIG. 3 proceeds to step S2. At step S2, lengths and cross-sections of the waveguides 103 of the array 102 are selected based on the desired shape of the ultrasound beam at the target 300 and on an ultrasound field produced by an ultrasound source 200 with which the device 100 is intended to be used. The lengths and cross-sections are selected such that the amplitude and/or phase of ultrasound emitted from each waveguide 103 of the array 102 provides the desired shape of the ultrasound beam at the target 300. For example, from a desired focal spot or focal pressure field at the target 300, time reversal or phase conjugation may be used to estimate a desired pressure field at an intended location of the device 100. Based on this desired field at the intended location, and the known field incident this intended location from the ultrasound source 200, the lengths and cross-sections of the device 100 can be determined. Properties of previously measured devices 100 of known cross-section and/or lengths may be used in to select the lengths and cross-sections.

As used herein, cross-section includes cross-sectional shape and/or cross-sectional size. The examples and results below primarily describe selecting cross-sectional size, with shape held constant. However it is to be appreciated that shape may also vary across the array 102.

To illustrate the ability of the length and cross-section of the waveguide 103 to determine a phase and/or amplitude ultrasound exiting that waveguide 103, ultrasound propagation through a waveguide 103 was modelled using k-wave, a pseudospectral method designed for weakly heterogenous media. K-wave is discussed in Treeby BE and Cox BT, k-Wave: MATLAB toolbox for the simulation and reconstruction of photoacoustic wave fields. J Biomed Opt. 2010; 15 (2): 021314, which is incorporated herein by reference.

FIG. 4 illustrates the k-wave simulation domain, showing the location of the structure 101, source plane, and measurement location. The k-wave software was used to model a repeating cell of the beam shaping device 100, comprising a waveguide 103 and surrounding structure 101. The cell was taken to have dimensions of approximately 3.4 mm×5.9 mm×2.7 mm, with the third dimension dependent on the thickness of the structure 101 (e.g. foam) being used. The cell was modelled using repeating boundary conditions in the directions planar with the structure 101 (x and y in FIG. 4), but a Perfectly Matched Layer (PML) was used above and below the structure 101. Results converged to constant values when the Courant-Friedrichs-Lewy number (CFL) was less than 0.1 and the number of Points per Wavelength (ppw) was greater than 35. For 500 kHz ultrasound, this meant a timestep of 10 μs and a spatial step of 9 μm. The simulation time was 57 μs, which took approximately 60 s on a desktop PC.

The ultrasound source 200 was modelled as an additive sound source on a uniform plane placed 1 mm from the foam grating, from which a pulse was emitted. The pulse had a duration of 13 μs and was band limited to 500 kHz.

The beam shaping device 100 was assumed to be in water as an ultrasound transmission medium. Water may typically be used as a transmission medium when the device 100 is used for a target 300 in a human body, to maximise transmission of ultrasound into the body by impedance matching. Water was modelled as a fluid with a sound speed of 1500 m/s and density of 1000 kg/m³. The waveguides 103 were taken to be holes in this example, and thus filled with the transmission medium (i.e. water) when in use. The structure 101 (foam) was modelled as a fluid with sound speed 1500 m/s and density 1.2 kg/m³. Foam actually has a density of 100 kg/m³, but as there was found to be negligible transmission through the structure 101 at 500 kHz, the sound speed is not known. The simulation values were therefore chosen in order to model a very low impedance material into which sound would not be readily transmitted, but with a sufficiently high sound speed to provide good numerical stability.

The transmitted ultrasound field was recorded on a plane 1 mm after the beam shaping device 100. Transmission coefficients were determined by taking the spatial average of the recorded ultrasound field, and comparing the Fourier transform of the transmitted field to the Fourier transform of the source field.

Results of this numerical simulation are shown in the dotted lines of FIG. 6, along with experimental results described further below. These results demonstrate that amplitude of the ultrasound can in some cases be determined primarily by the radius of the waveguides 103 (or in general, a cross-section of the waveguide or parameter thereof, including aspect ratio, smallest cross-sectional dimension of the waveguide 103 or a circumdiameter of a circle circumscribed by the cross-section of the waveguide 103). The phase can be determined by the length of the waveguide 103 (equivalent to the thickness of the foam structure 101 in the illustrated case) and/or the radius/cross-section. In the following sections the selection of cross-section of waveguides 103 is described primarily in terms of selecting diameters for circular waveguides 103, or equivalently selecting circumdiameters or smallest cross-sectional dimensions of non-circular waveguides 103. However it is to be appreciated than in general any aspect of the cross-section may be selected to yield the desired amplitude and/or phase. This may include the shape of the cross-section of the waveguide, an aspect ratio of the cross-section of the waveguide, and/or cross-sectional sizes such as a largest cross-sectional size or smallest cross-sectional size. Any of these aspects of cross-section may vary across the array 102, while other aspects are fixed for all waveguides 103.

This can be further confirmed by considering an analytical model of the waveguides 103. It is hypothesized that the foam holes will act as waveguides 103, despite their short length (1 mm-10 mm) relative to their diameter (1.5 mm-3 mm). Expressions may be obtained for ultrasound propagation inside a narrow tube, assuming the foam acts as a free surface.

A symmetric acoustic field inside a waveguide can be described by a velocity potential ϕ(r,z,t), from which the particle velocity is u(r,z,t)=∇ϕ and the pressure is

$$p ( r , z , t ) = - {ρ}_{0} \frac{\partial ϕ}{\partial t} .$$

Time harmonic waves propagating in the z direction can be described by:

$$ϕ = \tilde{ϕ} ( r ) \cdot {e}^{j ( {k}_{z} z - ω t )} \qquad ( 1 )$$

If the field is bounded at r=0, the only solution that is allowable in the hole is a zero order Bessel function given by:

$$\tilde{ϕ} = {J}_{0} ( r \sqrt{{k}^{2} - {k}_{z}^{2}} ) \qquad ( 2 )$$

The free surface at r=α enforces a boundary condition ϕ=0, in which case the term α\sqrt{k²-k_{z}²}=α₀ᵢ where α₀ᵢ is the ith zero of J₀(x). The lowest order mode is when α₀₁=2.4048, in which case:

$${k}_{z} = \sqrt{{k}^{2} - {( \frac{2.4048}{a} )}^{2}} \qquad ( 3 )$$

Propagation only occurs for frequencies high enough that the argument in the square root is positive, or alternatively for a given frequency ƒ there is a critical size that the hole must exceed in order for sound to propagate:

$${a}_{crit} > \frac{{α}_{01} c}{2 π f} \qquad ( 4 )$$

By using waveguides 103 similar in size to this critical radius, the ability of waves to propagate changes dramatically. Thus, in a waveguide of fixed length, the amplitude of the transmitted wave can be controlled. In addition, when the waveguide 103 radius is above the critical radius, the real part of the propagation constant k_{z} is dependent on the hole diameter. This means that the phase of the wave as it is transmitted to the end of the waveguide can also be changed. For non-circular waveguides 103, the equivalent of diameter (i.e. 2× radius) will be a cross-sectional size of the waveguide (i.e. a dimension of the waveguide 103 in a plane substantially orthogonal to the direction of propagation of the incoming ultrasound wave, optionally the size on an axis of a coordinate system in which the target beam shape is defined, such as the x or y axes in the illustrated figures) . . . . This may be a circumdiameter of the circle circumscribed by the cross-section of the waveguide 103, or a smallest cross-sectional dimension. For example, this may include width and/or height or cross-sectional area of the waveguide 103, particularly where the waveguide has a square or rectangular cross-section.

Equations 1-4 predict that for waveguides 103 in foam, the phase velocity inside the waveguide is a function of the waveguide radius/cross-sectional size, rather than just the material properties of the fluid inside it. This indicates that it should be possible to manipulate sound transmission using an array 102 of suitable waveguides 103 of varying radius.

Therefore in step S2, by appropriately selecting the lengths and cross-section (in particular smallest cross-sectional dimension e.g. diameter, or circumdiameter) of the waveguides 103, the phase and/or amplitude of ultrasound can be tailored to create a desired ultrasound field at a target 300.

At least one of the length and cross-section of the waveguides 103 may be fixed for all waveguides 103. In other cases the lengths and/or cross-sections may vary across the array (i.e. at least one waveguide 103 has a different length/cross-section to another waveguide 103 in the array). In the latter case, the cross-sectional shape of the waveguides 103 may be fixed, but the cross-sectional sizes vary across the array. Examples of arrays of varying cross-section and length are illustrated in FIGS. 5a and 5b, discussed further below.

In general, different lengths and/or cross-sections may be selected for each individual waveguide 103. Alternatively waveguides 103 may be grouped, with different lengths/and or cross-sections selected for each group of waveguides 103. Further alternatively all waveguides 103 may be selected to have the same lengths and/or cross-sections.

To demonstrate the control of ultrasound field possible with a beam shaping device 100, a beam shaping device 100 was constructed from a foam sheet to match the design shown in FIG. 1a. Holes of varying diameter were cut into an EVA foam sheet (Zunchuan Maoyi Ltd, Shanghai) using a diode laser cutter (Laser Master 2 15 W, Ortur, Changping, Guangdong).

A further factor controlling amplitude of the ultrasound at the target 300 is pitch of the waveguides. As used herein, pitch is the distance between the centre of a waveguide 103 and the centre of the next nearest waveguide. In particular, this may be measured from cross-sectional centres of the waveguides in a plane orthogonal to the axis of propagation of the ultrasound, preferably at the entrances to the waveguides. Preferably, the pitch of waveguides 103 is substantially the same for all waveguides 103 in the array 102, but in some examples pitch may vary across the array 102. Various values of pitch may be used, but a 3.4 mm centre to centre pitch (spacing) between holes was used for all experiments in this study. This has been found to be most suitable for 500 kHz ultrasound propagation, to maximise overall ultrasound transmission. In general, the optimal pitch will depend on the wavelength 2 of the ultrasound emitted by ultrasound source 200 with which the device 100 is intended to be used. In particular, the pitch (or average/mean pitch) may be in a range with a maximum value of 2λ, or preferably 1.5λ, or more preferably 1.2λ. The pitch (or average/mean pitch) may be in a range with a minimum value of 0.7λ, or preferably λ, or more preferably 1.1λ. The pitch (or average/mean pitch) may be 1.3λ. In particular examples, the pitch (or average/mean pitch) may be in range with a maximum value of 5 mm, or preferably 4 mm, or more preferably 3.5 mm. The pitch (or average/mean pitch) may be in a range with a minimum value of 2 mm, or preferably 3 mm, or more preferably 3.3 mm. Pitch between waveguides may be measured by any common method, such as using a micrometer. The pitch may also be selected in step S2 based on the desired beam shape at the target 300.

For the experimental results, as shown in FIG. 1a, waveguides 103 (i.e. holes in this case) were placed on a hexagonal grid to maximize the number of waveguides 103 at a given centre to centre pitch. The hexagonal array 102 was separated into sections based on waveguide diameter. Within a section, each waveguide 103 had approximately the same diameter. It is to be appreciated in general that any layout of waveguides may be used.

For the experiments, the ultrasound source 200 was a 19 mm diameter planar cylindrical transducer (NDT V318, Olympus IMS, MA, USA). This source 200 was used to insonate 60 mm×60 mm devices 100 placed 500 mm from the transducer in a custom made acrylic water tank (90 cm×56 cm×50 cm). This setup ensures that the sound incident on the gratings is approximately plane, with a phase deviation of 136°, and amplitude deviation of 30% in the field incident on the 60 mm×60 mm gratings. It is to be appreciated that in general any appropriate relative position of beam shaping device 100, ultrasound source 200, and target 300 may be used, with the parameters of the beam shaping device 100 selected accordingly. In particular, the spacing between the source 200 and device 100 may be such that ultrasound incident upon the device 100 approximates a plane wave, but this is not necessary. The device 100 may be positioned (or intended to be positioned) closer to the source 200. In some examples, the device 100 may be placed adjacent to the source 200, with negligible spacing between the device 100 and spacing 200. For example, the source 200 may comprise a plurality of ultrasound transducers, each positioned adjacent to a respective waveguide 103 of the device 100 to directly insonate individual waveguides 102. Further, although in these experiments the device 100 was placed in water, in general the device 100 may be positioned (or intended to be positioned) in any medium capable of transmitting ultrasound. The structure 101, or properties thereof, may be selected based on the intended medium, to ensure at least partial ultrasound reflection.

In the experiments, the transducer was driven at 500 kHz with 10 cycle sinusoidal pulses of 20 V amplitude by a signal generator (33250A, Keysight, Santa Rosa, CA, USA) and RF amplifier (325LA E&I, Rochester, NY, USA) every 20 ms. This led to a peak negative pressure of 3.9 kPa at the location where the device 100 was placed. The water in the tank was filtered, and at room temperature. Degassing was not deemed necessary due to the very low sound pressures used. The transmitted field was measured over a plane 10 mm past the grating with a 400 μm needle hydrophone (HNA-0400, Onda Corporation, Sunnyvale, CA, USA). Measurements were taken on a 54×54 grid at locations 1.3 mm apart (spatial extent 68.9 mm×68.9 mm), and waveforms were saved using a digital oscilloscope (WaveRunner 64Xi-A, LeCroy, Chestnut Ridge, NY, USA).

Using this set up, ultrasound transmission was measured through three different beam shaping device 100, having thicknesses (setting in this example a common waveguide 103 length within each device 100) of 1.5 mm, 3 mm and 5 mm. Each device 100 contained circular waveguides 103 with radii evenly spaced from 0.9 mm to 1.5 mm (separated into sections of common radius, as shown in FIG. 1a). Numerical simulations were performed for the same conditions, as discussed above.

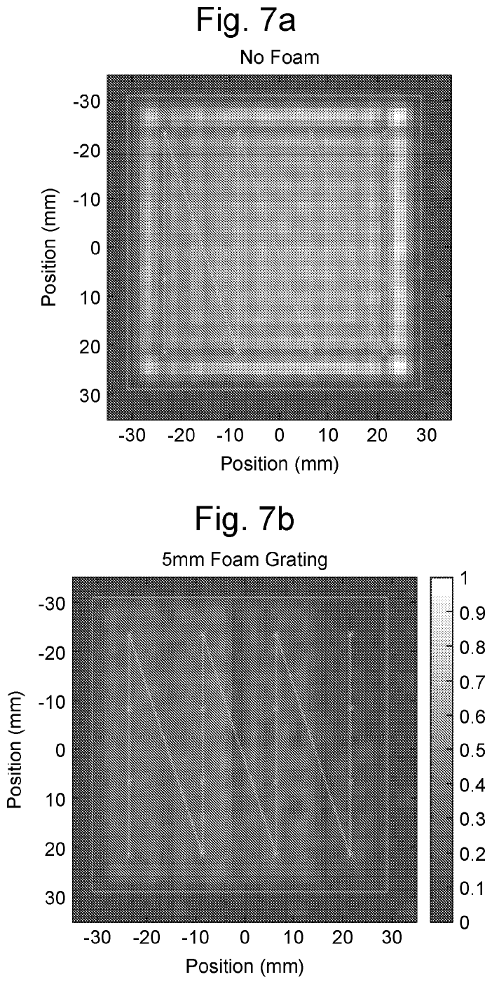

The scans were processed using Matlab (R2020A, The Mathworks, Natick, MA, USA). The complex value for the signal at every point at 500 kHz was extracted by using a 16384 (2¹⁴) point zero padded FFT. The Angular Spectrum Method (ASM) was used to estimate the complex pressure at the surface of the grating from the measurements made 10 mm past the grating. FIG. 7 shows an example of the measured data after being back propagated to the location of the device 100. The back propagated data were then spatially averaged over a 10 mm×10 mm area in the centre of the (9 or 16) square sections. This provided an average value for the amplitude and phase of pressure transmitted by every square section. FIG. 7a shows the result with no device 100 present, showing the approximately uniform sound field. FIG. 7b shows the sound field when a 5 mm thick foam device 100 with holes radii from 0.9 to 1.5 mm is used. The lines and markers show the centres of the square sections of different hole radii, and where the pressure for transmission through each square is sampled.

The process was repeated for the daily control scan with no device 100 present. The ratio of the complex pressures with a device 100 present compared to no grating gives the pressure transmission coefficient for all 16 sections. The measurement and data processing methods were intended to correct for diffraction after transmission through the device 100, any changes to the sound source or water from day to day, and the heterogeneity of the incident sound field.

The phase and amplitude of the transmitted ultrasound from both the experiments and simulations are shown in FIG. 5. Both linear and polar plots are shown. The polar plot indicates the phase and amplitude change that can be achieved by transmission through the beam shaping devices 100, but does not as readily show which hole radius leads to what effect.

All waveguide lengths were able to vary the amplitude of the transmitted ultrasound up to at least 75% by appropriate selection of waveguide radius. The longer waveguides 103 had a slightly higher threshold radius at which transmission increased, and a sharper transition to a maximum value. All three waveguide lengths exhibited a maximum transmission of around 80% when holes were larger than the critical radius. All gratings caused a phase change in the transmitted sound. For small radius holes, each device 100 caused a phase lead, which reduced as the radius of the holes increased. The size of the phase lead, and the rate of change of phase with respect to hole radius was larger for longer waveguide lengths.

The results of the k-wave numerical model are also shown in FIG. 6 and were in generally good agreement with the experimental results. The numerical model accurately predicted the phase in almost all cases, with significant deviations from the measurements only when the transmission amplitude was <20%. The numerical model also captured the main features of the amplitude changes, but consistently over-predicted its magnitude. In one of the simulations (5 mm foam), a transmitted amplitude of 107% is reported, which may indicate some numerical instability in the model.

The phase results from the experiment are duplicated in FIG. 7, with the addition of the predictions from the analytical waveguide model. The analytical model also showed close agreement with the experimental measurements, and in most cases the predictions were more accurate than those of the numerical model. This suggests the proposed mechanism for the phase change produced by the gratings is correct.

According to the waveguide model, when the waveguides 103 are smaller than a critical radius, transmission only occurs through evanescent waves. This predicts that there is a large phase lead, which should be directly proportional to the length of the waveguide 103. According to equation 3, as the waveguide 103 radius increases, the propagation constant for waves in the waveguides 103 becomes a small real value, and eventually approaches the propagation constant in water. This predicts that the phase lead caused by the holes will reduce to zero as the waveguide 103 radius increases.

These results demonstrate that by selecting the lengths and cross-sections of the waveguides, the ultrasound field/beam can be shaped as desired for a particular target 300.

In some cases, the desired field may comprise a desired phase of the wave at positions within the target, with a maximum possible amplitude at any given point in the target. In such cases, the cross-section may be fixed for all waveguides 103 (e.g. to a value less than the critical diameter/equivalent critical circumdiameter/size for non-circular cross-sections, such as a critical value of smallest cross-sectional dimension). In other cases, the desired field may include specific desired amplitudes at points within the target. In such cases, each waveguide 103 (or some waveguides 103) may have a different diameter/cross-sectional size. As used herein, amplitude includes a relative amplitude determined with respect to an amplitude of the ultrasound incident on the device 100).

FIG. 5a illustrates a cross-section through a part of a beam shaping device 100, showing two waveguides 103a, 103b of varying cross-sections but fixed lengths. In this example the beam shaping device 100 is shown as formed of a substrate, similar to FIG. 1b, but it is to be appreciated that the device 100 may take other forms, such as that shown in FIG. 1c. The smallest cross-sectional dimension (here diameter) of waveguide 103a is less than that of waveguide 103b. Other, not shown, waveguides may have cross-sections the same as one of waveguides 103a, 103b, or different still. Varying the cross-sections in this way may allow control of the amplitude, or phase and amplitude of the ultrasound beam at the target 300. It is to be appreciated that the lengths of the waveguides 103a, 103b may also vary.

As will be appreciated, the diameter/circumdiameter/smallest cross-sectional dimension of a waveguide 103 required to provide a particular amplitude will vary depending on the wavelength 2 of the ultrasound for which the device 100 is intended to be used, when in the waveguide 103 (including in the material of solid waveguides 103, or in the transmission medium filling holes acting as waveguides 103). In some examples the diameter/circumdiameter/smallest cross-sectional dimension of each waveguide 103 may selected from a range with a maximum value of 2. The diameter/circumdiameter/smallest cross-sectional dimension of each waveguide may be selected from a range with a minimum value of 0.62. The diameter/circumdiameter/smallest cross-sectional dimension of each waveguide 103 may be selected from a range with a maximum value of 5 mm. The diameter/circumdiameter/diameter/smallest cross-sectional dimension of each waveguide is selected from a range with a minimum value of 0.3 mm Such values may be appropriate for incident ultrasound of in the range 300 kHz-3 MHz. In particular examples, the diameter/circumdiameter/smallest cross-sectional dimension of each waveguide may be selected from a range with a maximum value of 3 mm. The diameter/circumdiameter/smallest cross-sectional dimension of each waveguide is selected from a range with a minimum value of 1.8 mm Such values may be appropriate for incident ultrasound of approximately 500 kHz. The diameters/circumdiameters/smallestcross-sectional dimensions may be measured by any common method, such as using a micrometer. As discussed above, such ranges allow the amplitude of the transmitted ultrasound to be tuned between the maximum and minimum possible values. For circular waveguides 103, the diameter may be selected based on the critical radius given by equation 4 above. For example, where amplitude is to be maximised, the diameter may be selected to be greater than the critical radius. For non-circular waveguides, the smallest cross-sectional dimension or circumdiameter may be selected based on an equivalent critical value.

In some cases, the desired field at the target 300 may comprise a desired amplitude of the wave at positions within the target, without any phase adjustment to the initial ultrasound wave, or with the phase free to vary. In such cases, a fixed waveguide length may be used for all waveguides 103. The common waveguide length may be set by the thickness of the structure 101, especially where the structure 101 is a substrate such as foam. In other cases, the desired field may include specific desired phase at points within the target (which may include a requirement that the phase is the same as that of the wave initially incident upon the device 100). In such cases, each waveguide 103 (or some waveguides 103) may have a different length.

FIG. 5b illustrates a cross-section through a part of a beam shaping device 100, showing two waveguides 103a, 103b of varying lengths but the same cross-section. In this example the beam shaping device 100 is shown as formed of a substrate, similar to FIG. 1b, but it is to be appreciated that the device 100 may take other forms, such as that shown in FIG. 1c. The length of waveguide 103a is less than that of waveguide 103b. Other, not shown, waveguides may have lengths the same as one of waveguides 103a, 103b, or different still. Varying the cross-sections in this way may allow control of the amplitude, or phase and amplitude of the ultrasound beam at the target 300. It is too be appreciated that the cross-sections (in particular the cross-sectional sizes) of the waveguides 103a, 103b may also be varied.

In some examples, the length of each waveguide may be selected from a range with a maximum value of 5λ. The length of each waveguide may be selected from a range with a minimum value of λ/3. In some examples, the length of each waveguide may be selected from a range with a maximum value of 15 mm. The length of each waveguide may be selected from a range with a minimum value of 1 mm. Waveguide length may be measured by any common method, such as using a micrometer.

As shown in FIG. 6, amplitude is primarily determined by the cross-sectional size/diameter. Thus in some cases cross-section may primarily be selected based on desired amplitude at the target 300. As also shown in FIG. 6, waveguide length has a large impact on the phase of ultrasound passing through the waveguides 103 (especially when the radius/size is less than the critical radius/size). Thus in some case length may primarily be selected based on a desired phase at the target 300. In the case of phase, however, there may also be a notable dependence on diameter/cross-sectional size (especially above the critical radius). Thus preferably both length and cross-sectional size are selected based on desired phase. In general, both length and cross-sectional size may be selected for either of amplitude and phase.

To further demonstrate the ability of the method of FIG. 3 to design a waveguide capable of generate a desired phase at a target 300 (or desired phases at spatial positions across the target 300), a beam shaping device 100 was designed to maximize phase change and transmission amplitude. As shown in FIG. 6, longer waveguide lengths allow the phase to be tuned fully by selecting an appropriate diameter/cross-sectional size. Although the waveguide length alone allows some tuning of phase, in general it may provide for easier construction if all waveguides have the same length, selected based on the desired phase tuning range, with phase tuning then done using changes in cross-sectional size/diameter.

To achieve maximum phase change, thicker foam was used, and the range of hole radii were restricted to above the critical radius (α_{crit}) in order to maximize transmission. The device 100 was constructed from two identical pieces of 5 mm foam stacked together. Each piece of foam could be considered as a separate layer of the structure 101, with the waveguides formed through both layers. Advantageously, this allowed simple laser cutting to be used to cut the holes in each sheet, whereas more difficult or less accurate techniques would be required to cut holes in a single thicker foam sheet. Similarly to the previous experiment, the grating was sectioned into 16 squares, each containing a different hole size spaced equally from 1.2 mm-1.66 mm

To provide a comparison of the performance of the device 100, two acoustic lenses were produced from PDMS (10:1, Sylgard 184, Dow, Midland, MI, USA) and from a photopolymer resin (Visijet M2R-TN resin, using an MJP 2500 Plus printer, 3D Systems, Rock Hill, SC, USA). A CAD model of the resin lens is shown in FIG. 9, and the PDMS lens used a similar geometry. The 60 mm×60 mm area was divided into 16 sections in the same way for the lenses and for the beam shaping devices 100. The thickness of the lenses was chosen to give a phase change of approximately 360° when the lenses were used in water.

The results from these lenses are shown in FIG. 10, alongside the results from the foam grating optimized for phase change. On the polar plot of transmission and phase, the data points for the lenses made from these two materials spiral slightly inwards as their thickness is increased, and attenuation reduces the transmitted amplitude. They spiral in different directions as the sound speed of PDMS (1030 m/s¹⁰) is less than that of water, and the sound speed of the resin (2500 m/s) is greater than that of water. Both lenses were able to generate any required phase change with more than 75% transmission. The beam shaping device 100 was able to generate phase changes of greater than 180° with 50% transmission, although transmission was reduced for larger phase changes.

These results show that the beam shaping device 100 can provide the phase control of an acoustic lens, even when constructed from holes in a foam sheet. Unlike acoustic lenses, the beam shaping device also allows control of amplitude. Thus the beam shaping device 100 of the present invention can be used to provide a desired beam shape for a particular target. FIG. 10 shows that by careful selection of waveguide length (which may be determined by the thickness of structure 101) and cross-section/diameter, it is possible to modify the phase of transmitted ultrasound with a limited reduction in amplitude. The phase change was increased by using thicker foam, and hence increasing the length of the waveguides. It may be possible to get a wider range of phase changes with smaller losses by further increasing material thickness and using larger diameter/cross-sectional size waveguides, by optimizing the shape to maximize sound entering into holes, and/or by using a structure material (e.g. foam) with a lower density and with less wetting.

In the experimental example described above, two foam sheets were combined to form the beam shaping device 100, with a longer effective waveguide length. In this case, both foam sheets had waveguides 103 of matching diameters and lengths. However, this need not be the case.

FIG. 11 shows a cross-section through a further example of a beam shaping device 100. The structure 101 of beam shaping device 100 comprises a first layer 101-1 and a second layer 101-2. The waveguides 103 (only one of which, 103a is labelled in the figure) extend through both layers 101-1, 101-2. As will be appreciated, the structure 101 may comprise any number of layers, but only two are described here for simplicity.

The device 100 of FIG. 11 is an example of a device 100 in which at least one waveguide 103 varies in cross-section along its length. In some cases this may comprise a tapering, but in the illustrated example the cross-section changes in a step change between the two layers 101-1, 101-2. A first part of a waveguide 103a-1 has a first cross-section, and a second part 103a-2 of the waveguide 103a has a second cross-section. In this case the cross-sectional size in the first layer 101-1 is larger than the cross-sectional size in the second layer 101-2, but this need not be the case. Other waveguides 103 may vary along their length in the same way, or, as shown in FIG. 11, may vary in different ways.

In the illustrated example, the respective lengths of the waveguides 103 differ in the two layers 101-1, 101-2. In this case the length of the first part 103a-1 of waveguide 103 is longer than the length of the second part 103a-2 of waveguide 103. In the illustrated example the lengths of all waveguides 103 within each layer 101-1, 101-2 are the same, but this need not be the case.

Advantageously, the lengths and cross-sections selected for the waveguides in the first layer 101-1 (e.g. of first part 103a-1 of waveguide 103) are selected primarily to provide control of the phase. The lengths and cross-sections selected for the waveguides in the second layer 101-2 (e.g. of second part 103a-2 of waveguide 103) are selected primarily to provide control of amplitude (or vice versa). This provides greater tuning of both amplitude and phase, without having to compromise between the two as may be the case for a single waveguide array 101.

In general, the lengths and/or cross-sections of any waveguide 103 or groups thereof may be varied in any way appropriate for the desired beam shape, as is the case for devices 100 formed of single layered structures. Although shown in the figure as directly connected, the various layers 102-1, 102-2 may be spaced part in use, so that ultrasound propagates through a transmission medium between the arrays 102-1, 102-2. Where directly connected, the respective layers 101-1, 102-2 may be physically attached, for example with an adhesive. The layers 101-1, 102-2 may be formed of the same material, or may be formed of different materials. Alternatively the layers 101-1, 102-1 may be formed of a single continuous structure, in which the waveguides 103 are formed.

A beam shaping device 100 of the form of FIG. 11 was constructed to demonstrate modulation of both amplitude and phase. The device 100 was constructed by placing a 1.5 mm foam grating on top of a 5 mm foam grating, such that the holes lined up and the foam pieces were in contact. Based on the predictions of the numerical model and earlier experiments, it was expected that thicker gratings with hole sizes from 1.09 mm-1.53 mm would cause a large phase change with limited change in amplitude, while thinner gratings with smaller hole sizes of 0.8 mm-1.5 mm could alter the transmitted amplitude with a lesser effect on phase. FIG. 12 shows the results for the composite device 100. The results demonstrate the feasibility of generating a range of phase changes and transmission amplitudes. The transmission amplitude is as high as 68%, and for transmission amplitude greater than 40%, this system was able to vary phase over 150°.

Returning to the method of FIG. 3, once beam shaping device 100 has been designed by selecting the lengths and cross-sections for the waveguides 103 (for example based on any of the exemplary devices 100 described above), the method may optionally proceed to step S3. At step S3, the beam shaping device is fabricated by forming a waveguide array into a structure in accordance with the selected parameters.

FIG. 13 illustrates a method of forming a beam shaping device 100 for shaping an ultrasound beam from an ultrasound source 200, which may be used as part of step S3 of the method of FIG. 3. The method of FIG. 13 starts at step S11, at which a structure 101 (or structures 101-1, 102-1) capable of reflecting ultrasound is provided. The method then proceeds to step S12, where lengths and cross-sections of the waveguides 103 are selected, or retrieved from the selection in step S2 of the method of FIG. 3. The method then proceeds to step S13, where the waveguide array 102 is formed in the structure 101 based on the selected parameters. As discussed above, forming the waveguide array 102 may comprise forming holes in the structure. Forming the waveguide array may comprise forming a plurality of pillars of ultrasound transmitting material and positioning the plurality of pillars in the structure.

1. A method of designing a beam shaping device for shaping an ultrasound beam from an ultrasound source, the beam shaping device comprising an array of waveguides formed in a structure that is capable of reflecting ultrasound, the method comprising:
determining a desired shape of the ultrasound beam at a target;
selecting lengths and cross-sections of the waveguides of the array based on the desired shape of the ultrasound beam and on an ultrasound field produced by an ultrasound source such that the amplitude and phase of the ultrasound emitted from the waveguides of the array provides the desired shape of the ultrasound beam at the target.

2. The method of claim 1, wherein the waveguides are holes formed through the structure.

3. The method of claim 1, wherein the waveguides are pillars of material capable of transmitting ultrasound.

4. The method of claim 3, wherein the material capable of transmitting ultrasound is a plastic.

5. The method of claim 1, wherein the structure is a substrate.

6. The method of claim 5, wherein the substrate is formed of a foam.

7. The method of claim 1, wherein the structure comprises gas held in a container.

8. The method of claim 1, wherein the waveguide array is designed for use in an ultrasound transmission medium, and wherein the ratio of acoustic impedance of the structure to the acoustic impedance of the transmission medium is 1/100 or less, or 1/500 or less.

9. The method of claim 1, wherein the structure has an acoustic impedance of 20000 Rayl or less, or 15000 Rayl and/or wherein the structure has an acoustic impedance of 400 Rayl or more, or 1000 Rayl or 1800 Rayl or more.

10. The method of claim 1, wherein the waveguides have varying lengths and/or cross-sections across the array.

11. The method of claim 1, wherein the waveguides have the same cross-sectional shape and have varying cross-sectional sizes across the array to provide varying cross-sections.

12. The method of claim 1, wherein a smallest cross-sectional dimension of each waveguide is in a range with a maximum value of λ, where λ is the wavelength of the ultrasound from the ultrasound source in the waveguide.

13. The method of claim 1, wherein a smallest cross-sectional dimension of each waveguide is in a range with a minimum value of 0.6λ, where λ is the wavelength of ultrasound from the ultrasound source in the waveguide.

14. The method of claim 1, wherein a smallest cross-sectional dimension of each waveguide is selected from a range with a maximum value of 5 mm.

15. The method of claim 1, wherein a smallest cross-sectional dimension of each waveguide is in a range with a minimum value of 0.3 mm.

16. The method of claim 1, wherein the length of each waveguide is in a range with a maximum value of 25 mm.

17. The method of claim 1, wherein the length of each waveguide is in a range with a minimum value of 0.5 mm.

18. The method of claim 1, wherein the average pitch is in a range with a maximum value of 2λ, or preferably 1.5λ, or more preferably 1.2λ, where λ is the wavelength of ultrasound from the ultrasound source in the waveguide.

19. The method of claim 1, wherein the average pitch is in a range with a minimum value of 0.7λ, or preferably λ, or more preferably 1.1λ, where λ is the wavelength of ultrasound from the ultrasound source in the waveguide.

20. The method of claim 1, wherein the average pitch is in a range with a maximum value of 5 mm, or preferably 4 mm, or more preferably 3.5 mm.

21. The method of claim 1, wherein the average pitch is in a range with a minimum value of 2 mm, or preferably 3 mm, or more preferably 3.3 mm.

22. The method of claim 1, wherein one or more waveguides have cross-sections varying along their length.

23. The method of claim 22, wherein the structure comprises a first layer and a second layer, wherein the waveguides are formed through each of the first layer and the second layer, and wherein the cross-section of at least one of the waveguides varies between the first layer and the second layer.

24. The method of claim 23, wherein each waveguide has a first length in the first layer and a second length in the second layer, and wherein for at least one waveguide the first length is different to the second length.

25. The method of claim 23, wherein
the lengths and cross-sections of the waveguides in the first layer are selected based on a desired one of amplitude or phase of ultrasound at the target; and
the lengths and cross-sections of the waveguides in the second layer are selected based on the other of amplitude or phase of ultrasound at the target.

26. The method of claim 1, wherein the ultrasound source emits ultrasound with a frequency of 3 MHz or less.

27. The method of claim 1, wherein the ultrasound source emits ultrasound with a frequency of 0.3 MHz or more.

28. The method of claim 1, wherein the ultrasound source comprise one or more ultrasound transducers.

29. The method of claim 1, wherein the target is a human body or is within a human body.

30. The method of claim 29, wherein the target is a tumour, or wherein the target is a region of the body comprising one or more tumours.

31. The method of claim 29, determining the desired shape of the ultrasound beam at the target comprises receiving measurements of the target in a patient.

32. (canceled)

33. A computer readable medium storing a computer program comprising instructions which, when executed by a computer, cause the computer to perform the method of claim 1.

34. A method of forming a beam shaping device for shaping an ultrasound beam from an ultrasound source, the method comprising:
providing a structure capable of reflecting ultrasound;
performing the method of claim 1 to select the lengths and cross-sections of waveguides of a waveguide array; and
forming the waveguide array in the structure based on the determined parameters.

35. The method of claim 34, wherein forming the waveguide array comprises forming holes through structure, each hole to act as a waveguide of the waveguide array.

36. The method of claim 34, wherein forming the waveguide array comprises:
forming a plurality of pillars of ultrasound transmitting material, each of the plurality of pillars to act as a waveguide of the waveguide array; and
positioning the plurality of pillars in the structure.

37. A beam shaping device for shaping an ultrasound beam from an ultrasound source, the beam shaping device comprising an array of waveguides formed in a structure that is capable of reflecting ultrasound, wherein the waveguides have lengths and cross-sections selected such that the amplitude and phase of the ultrasound emitted from the waveguides of the array provides a desired shape of an ultrasound beam at a target.

38-61. (canceled)

62. A method of shaping an ultrasound beam from an ultrasound source, the method comprising:
positioning a beam shaping device according to a claim 37 between the ultrasound source and a target such ultrasound from the ultrasound source is incident upon the waveguide array of the beam shaping device, and such that ultrasound exiting the waveguide array forms a desired ultrasound beam at the target.
